(19) United States
(12) Patent Application Publication
(10) Pub. No.: US 2023/0316719 A1
(43) Pub. Date: Oct. 5, 2023

(54) AUTOMATIC DETECTION OF DENTAL INDICATIONS

(71) Applicant: Ivoclar Vivadent AG, Schaan (LI)

(72) Inventors: Alen Frey, Feldkirch (AT); Marius Aster, Kastellbell/Tschars (IT); Hendrik John, Buchs (CH)

(21) Appl. No.: 18/193,550

(22) Filed: Mar. 30, 2023

(30) Foreign Application Priority Data
Mar. 31, 2022 (EP) 22165854.5

(51) Int. Cl. G06V 10/764; G16H 30/40; G06V 10/82; G06V 20/64

(52) U.S. Cl. CPC G06V 10/764; G16H 30/40; G06V 10/82; G06V 20/64; G06V 2201/03

(57) ABSTRACT

A recognition method for a dental object, including the steps of providing (S101) a digital dental object in a coordinate system describing a shape of the dental object to be manufactured; and automatically assigning (S102) the digital dental object to a predetermined class based on the shape by a self-learning algorithm.

### CROSS-REFERENCE TO RELATED APPLICATIONS

This application claims priority to European Patent Application No. 22165854.5 filed on Mar. 31, 2022, the disclosure of which is incorporated herein by reference in its entirety.

### TECHNICAL FIELD

The present invention relates to a dental object recognition method, a dental object manufacturing apparatus, and a computer program for recognizing a dental object.

### BACKGROUND

In known digital workflows, dental restorations are generated in the CAD software and saved in a 3D CAD file format. Depending on the used CAD software and the subsequent workflow, so-called metadata can be generated and stored together with the 3D CAD file, which includes additional information.

Metadata can be helpful to control or optimize the ablative manufacturing methods, such as assigning milling strategies for a specific restoration type. In three-dimensional printing methods in which support structures are required, such as in stereolithography, VAT polymerization, 3D DLP printing, or selective laser sintering of metals, certain surfaces of dental restorations should be free from the connection of support structures in order to positively influence precision and surface quality.

In the case of metadata, however, there is the disadvantage that these are already generated in the CAD software beforehand and that these are a proprietary data format. In this case, any CAM software used must be able to import and interpret the associated proprietary metadata. Since these metadata formats are not universal, a separate interface must be programmed for each proprietary metadata format. If no metadata is available for an object, the CAM software cannot determine which indication it is during import.

However, the indication is important for determining which manufacturing method should be used to produce a dental object. The classification can also be used in further data processing and for determining further method steps for manufacturing or for material processing in the digital workflow.

U.S. Pat. No. 10,856,957 B2 relates to a method of producing a three-dimensional digital model of a prosthetic base for fabrication using a light-based three-dimensional printing device, and is hereby incorporated by reference in its entirety. US 20210255600 is directed to a method of producing a dental restoration and is hereby incorporated by reference in its entirety.

US 20070048689, 20090319068, 20200000562, 20200179082, 20060008774, U.S. Pat. Nos. 10,838,398, 10,722,974, 9,939,806, 8,655,628, 8,483,857, 8,214,178, and 8,209,044, are directed to methods and materials for making dental restorations and are hereby incorporated by reference in their entirety. U.S. Pat. Nos. 10,915,934, 10,871,764, and 10,882,303, are directed to methods/machines using computers in carrying out various processes and are hereby incorporated by reference in their entirety.

### SUMMARY

It is the technical object of the invention to identify the indication of digital dental objects based on the geometric shape, so that a suitable manufacturing method can be selected.

This technical object is solved by subject-matter according to the independent claims. Technically advantageous embodiments are the subject of the dependent claims, the description, and the drawings.

According to a first aspect, the technical object is solved by a recognition method for a dental object, comprising the steps of providing a digital dental object in a coordinate system describing a shape of the dental object to be manufactured; and automatically assigning the digital dental object to a predetermined class based on the shape by a self-learning algorithm. The class may be associated with a specific manufacturing method that can be used to manufacture the real dental object based on the digital dental object. Depending on the class of the dental object, adapted manufacturing methods with optimal parameters can be used.

In a technically advantageous embodiment of the recognition method, a number of points are detected on the surface of the digital dental object. This achieves, for example, the technical advantage that the digital dental object can be quickly assigned with a small amount of classification data.

In a further technically advantageous embodiment of the recognition method, the points on the surface of the digital dental object are selected randomly. This has the technical advantage, for example, that the population of classification data can be obtained without complicated calculations.

In a further technically advantageous embodiment of the recognition method, the coordinates of the detected points form an input for an artificial neural network. This achieves, for example, the technical advantage that the digital dental object can be efficiently assigned to a class.

In a further technically advantageous embodiment of the recognition method, the artificial neural network has been trained by a plurality of training data sets. This also achieves the technical advantage, for example, that the digital dental object can be efficiently assigned to a class.

In a further technically advantageous embodiment of the recognition method, the class of the digital dental object is output by the artificial neural network. This also achieves the technical advantage, for example, that the digital dental object can be efficiently assigned to a class.

In a further technically advantageous embodiment of the recognition method, a digital reference object is assigned to the digital dental object based on the assigned class. This has the technical advantage, for example, that a workflow can be further optimized.

In a further technically advantageous embodiment of the recognition method, the digital dental object is transformed in the coordinate system based on the assigned class and/or the reference object. The digital dental object can, for example, be aligned with the reference object or moved to the reference object. This achieves the technical advantage, for example, that the manufacturing method can be carried out with an adapted orientation and/or position of the digital dental object.

In a further technically advantageous embodiment of the recognition method, a manufacturing method is assigned to the digital dental object based on the assigned class. This has the technical advantage, for example, that the dental object can be manufactured efficiently, and the manufacture of different dental objects can be automated.

In a further technically advantageous embodiment of the recognition method, further spatial structures are added to the digital dental object based on the associated manufacturing method. The spatial structures can be support structures for a build platform or holding structures for a blank. This achieves, for example, the technical advantage that the manufacturing of the dental object can be further improved.

In a further technically advantageous embodiment of the recognition method, the dental object is produced by the manufacturing method. This provides, for example, the technical advantage that the dental object is manufactured using a suitable manufacturing method.

In a further technically advantageous embodiment of the recognition method, the manufacturing method is an additive or subtractive manufacturing method. This achieves, for example, the technical advantage of using particularly suitable manufacturing methods for dental objects.

In a further technically advantageous embodiment of the recognition method, a correctness of the assignment is checked by geometric features of the digital dental object. This has the technical advantage, for example, of improving the accuracy of the method.

According to a second aspect, the technical object is solved by a manufacturing device for a dental object, which is adapted to perform the recognition method according to the first aspect. By the manufacturing device the same technical advantages are achieved as by the recognition method.

According to a third aspect, the technical problem is solved by a computer program comprising instructions which, when the computer program is executed by a computer, cause the computer program to execute the method according to the first aspect. By the computer program the same technical advantages are achieved as by the recognition method.

It is preferable that a computer program product includes program code which is stored on a non-transitory machine-readable medium having computer instructions executable by a processor, which computer instructions cause the processor to perform the recognition method described herein.

### BRIEF DESCRIPTION OF THE DRAWINGS

Examples of embodiments of the invention are shown in the drawings and are described in more detail below.

The figures show:

FIG. 1 a schematic representation of a classification method;

FIG. 2 a plurality of points from a classification data set;

FIG. 3 a schematic view of an artificial neural network;

FIG. 4 digital dental objects of different classes;

FIG. 5 a display of a recognized class; and

FIG. 6 a block diagram of a recognition method for a dental object.

### DETAILED DESCRIPTION

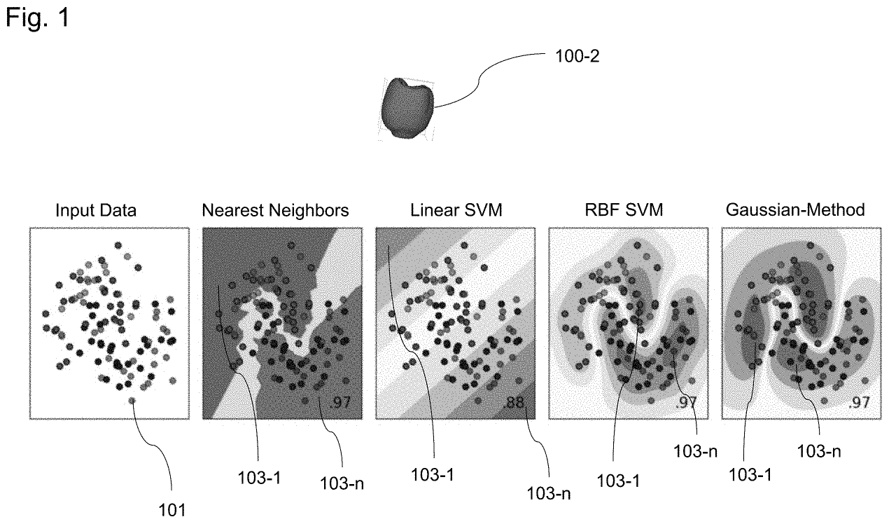

FIG. 1 shows a schematic representation of classification methods. For example, a real dental object 100-2 is a crown, bridge, veneer, abutment, inlay, onlay, splint, or partial or full denture. In general, dental object 100-2 can be any object in the dental field that is to be additively or subtractively manufactured as part of a three-dimensional manufacturing method.

For each of these real dental objects 100-2, there may exist a digital dental object 100-1 in which the three-dimensional shape and the color values are specified. This information is stored, for example, in a data set for the digital dental object 100-1.

With the aid of a classification of the digital dental object 100-1, it can be determined to which type it belongs. For example, the type of digital dental object 100-1 can be “crown”, “bridge”, “veneer”, “abutment”, “inlay”, “onlay”, “splint” or “partial denture” or “full denture”. In general, the type of digital dental object 100-1 can be any type by which real dental objects 100-2 can be distinguished from each other.

For example, differentiation of the digital dental objects 100-1 may be performed based on their respective shapes. Thus, classification may be performed based on classification data sets 101 comprising a plurality of points on the surface of the digital dental object 100-1. If numerous points 115 on the surface of the digital dental object 100-1 are known, it can be automatically determined, for example, whether the object is an abutment or a full denture.

Each point in the graphs represents a single classification data set 101 for a digital dental object 100-1 as input data. In a nearest neighbor classification, a nonparametric method for estimating probability density functions is performed for the classification data sets 101. The resulting K-nearest-neighbor algorithm is a classification procedure in which class assignment is performed considering its k nearest neighbors.

In a classification with a linear Support Vector Machine—SVM (support vector machine) for the classification data sets 101, a classifier and a regressor are used for the regression analysis. The Support Vector Machine divides the set of digital dental objects into classes in such a way that the widest possible range of objects remains free around the class boundaries. The Support Vector Machine is a so-called large margin classifier.

Additionally, a radial basis function (RBF) can be used, whose value depends on the distance to the origin. In a classification with a Gaussian method, a stochastic process is used for the classification data sets 101, in which each finite subset of random variables is multidimensionally normally distributed (Gaussian distributed).

Each classification data set 101 can be assigned to a class 103-1, . . . , 103-n by the classification method, such as whether it is the class “abutment” or the class “full prosthesis”. The manufacturing method for a real dental object 100-2 can be finally determined based on the class of the digital dental object 100-1.

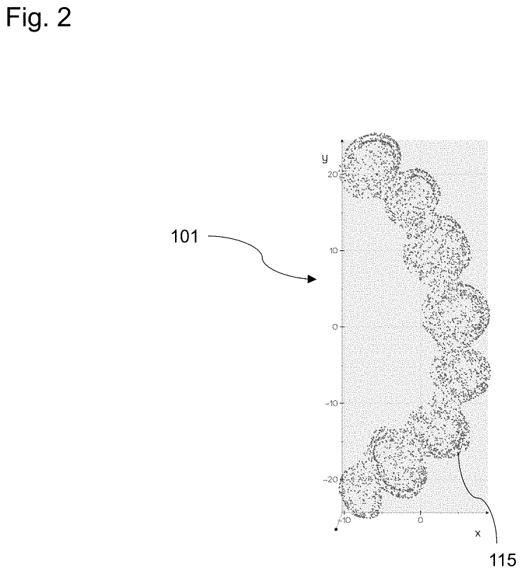

FIG. 2 shows a plurality of points 115 on the surface of the digital dental object 100-1, which may be present in a classification data set 101. The points 115 are each defined by coordinates X, Y, and Z of a coordinate system.

This classification data set 101 is assigned to the class “full prosthesis” by the classification procedure. To make the classification procedure invariant to an input permutation, symmetric functions are used. A symmetric function is a function where the variables can be interchanged without changing the function value.

The classification method can be trained using a plurality of classification data sets 101 of which the respective class is known. New classification data sets 101 can then be classified based on their similarity to the trained classification data sets 101.

In the classification method, depending on the input data and its properties, the classification algorithm independently learns a suitable similarity measure during training to distinguish the different classes of dental objects 100-1. Therefore, it is not necessary to define an explicit similarity measure. The classification algorithm, like a neural network for example, implicitly determines itself during the training phase based on the training data which properties of the point clouds are relevant to map a similarity. This is then used to classify the classification data set 101. Depending on the shape of the training data set, the similarity measure may vary. This is an effect that generally occurs with more complex classification algorithms and is deliberately accepted.

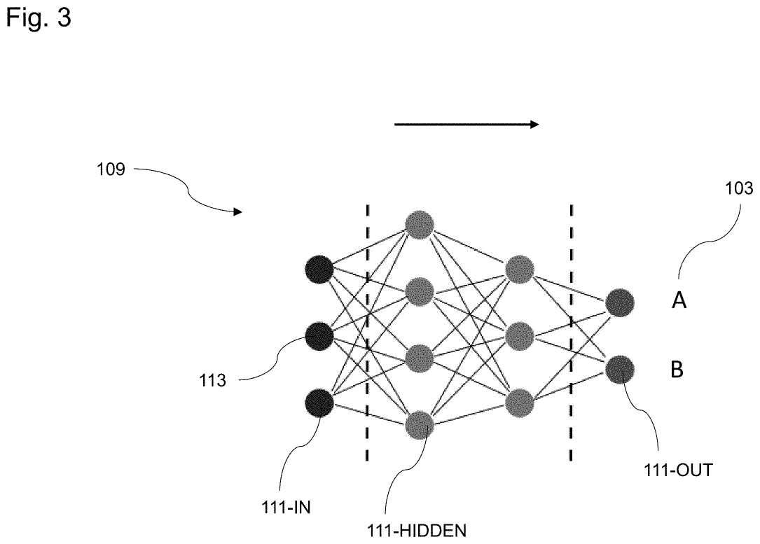

FIG. 3 shows a schematic view of an artificial neural network 109. The artificial neural network 109 is a network of artificial neurons and can be used to classify the digital dental object 100-1. The artificial neural network 109 includes an input layer 111-IN having a number of neurons 113 corresponding, for example, to the number of points 115 on the surface of the digital dental object 100-1 in the classification data set 101. In this case, each point from the classification data set 101 is input to a separate neuron 113.

These are forwarded to the neurons 113 of hidden layers 111-HIDDEN. Thereby an individual weighting of each signal from one neuron 113 to another neuron 113 takes place. Subsequently, the result is output at the output layer 111-OUT. For example, the number of neurons 113 at the output layer 111-OUT corresponds to the number of classes. In this way, a mapping is created:

F(X₁,Y₁,Z₁, . . . ,Xₙ,Yₙ,Zₙ)->class₁, . . . ,classₘ

When the neural network 109 is trained, a plurality of classification data sets 101 of which the respective class is known are fed. The neural network 109 learns by modifying the weights between the neurons 113, adjusting the weights until the output class corresponds to that class which is known for the classification data set 101.

In general, a combination of convolutional layers (convolutional layer) and fully connected layers (dense layer) can be used. Sigmoid, Tan h or ReLU functions can be used as activation functions. Batch normalization can be performed after each layer.

For example, the invention can be implemented by the following source code:

from tensorflow import keras
from lib import *
import numpy as np
import trimesh
import tensorflow as tf
# load map of currently trained classes
CLASS_MAP = load_dict(CLASS_MAP_FILE_PATH)
# load neural net
model = keras.models.load_model(“saved_models/model”)
# load stl file
mesh = trimesh.load(“example.stl”)
points = mesh.sample(2048) # sample point cloud of 2048
points from the mesh
points = np.expand_dims(points, axis=0) # expand dimension
to create a batch
# to classify multiple files, just add more point sets to
the batch
# get classification of the model
p = model.predict(points)
p = tf.math.argmax(p, −1)
p = CLASS_MAP[str(p[0].numpy( ))] # get string label of
classification
print(p) # show class

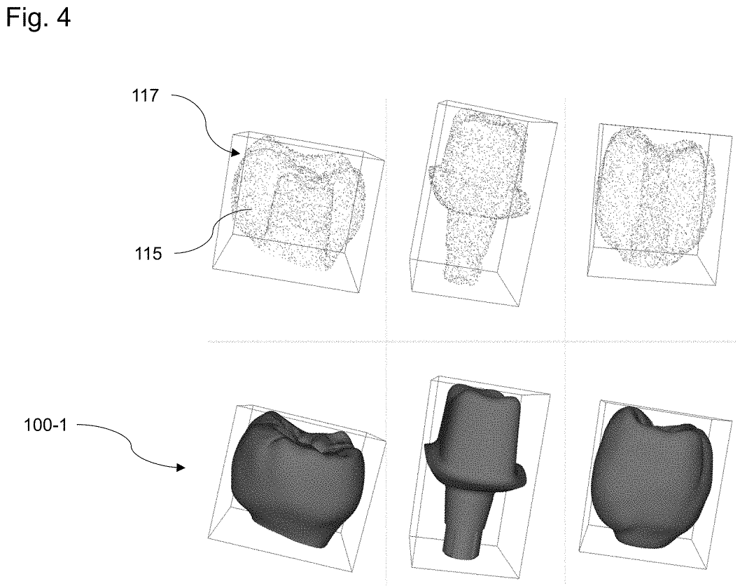

FIG. 4 shows several digital dental objects 100-1 of different classes, such as a crown (left), an abutment (center), and another crown (right). The surface of these dental objects 100-1 is described by a point cloud 117 of random points.

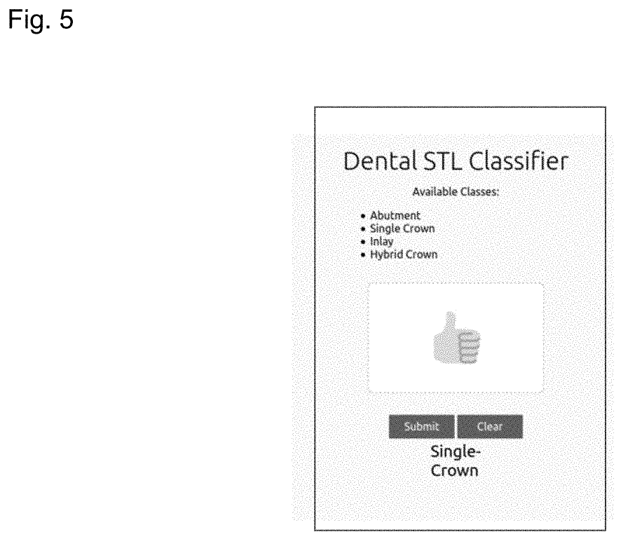

FIG. 5 shows a display of a detected class. The class of the digital dental object 100-1 may be displayed. Then, in turn, a unique identifier may be added to the data set of the digital dental object 100-1. Thus, an initially unknown data set may become a data set that is appropriately classified and may again be used for training the classification algorithm.

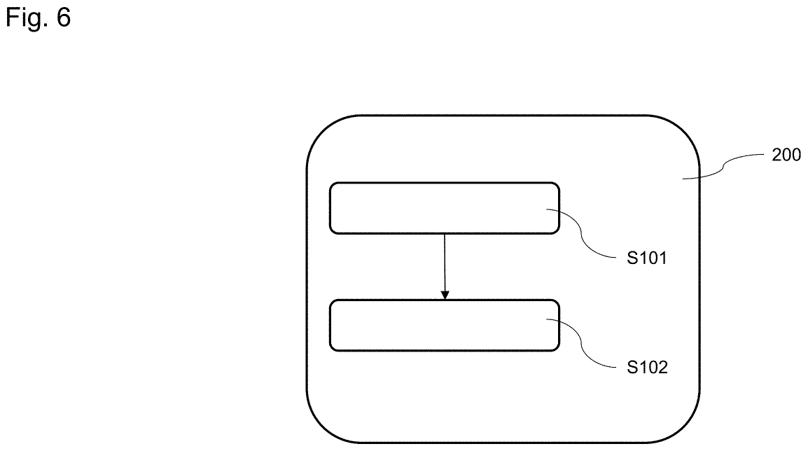

FIG. 6 shows a block diagram of a recognition method for a dental object 100-1. The recognition method comprises the step S101 of providing the digital dental object 100-1 in a coordinate system that describes a shape of the dental object 100-2 to be manufactured. For this purpose, for example, the 3D dental data set of the digital dental object 100-1 is imported. Then, a plurality of uniformly distributed random points on the surface of the dental object 100-1 may be determined, such as 4096 random points, and stored in a classification data set 101.

Subsequently, in step S102, the digital dental object 100-1 is automatically assigned to a predetermined class based on the shape by a self-learning algorithm. For this purpose, this algorithm has been previously trained with a plurality of known digital dental objects 100-1 and their respective dental classification and/or indication. Each indication is a subset of a class. The self-learning algorithm can optimize the parameters of a function that maps the geometric information of the digital dental object 100-1 to the respective class.

The recognition method allows the identification of the three-dimensional data sets for the digital dental objects 100-1 to be performed automatically and without attached metadata when imported into the CAM software. No static algorithm is used to explicitly identify specific features of the digital dental object 100-1, but a dynamic machine learning algorithm from which the features are learned implicitly.

The result depends on the three-dimensional structure and the number of available digital dental objects 100-1 with an indication that have been used for training. Then, in a further step, a verification and plausibility check of the classification can be performed using simple geometric features, such as based on dimensions of the digital dental object 100-1 (bounding box, centroid, local curvature).

The plausibility check is performed after classification, when the classification result has already been determined, in order to check it again. For example, the volume of the dental object 100-1 can be used to check the plausibility. The volume can be used, for example, to check whether the previous classification result “single crown” is correct. The volume can be used to check whether it is actually a single crown or whether the volume is too large for a single crown and rather corresponds to the volume of a bridge.

Furthermore, features such as the dimensions of a bounding box, a maximum size of the bounding box, the aspect ratios of the bounding box, a curvature of the dental object or a position of the center of mass of the dental object can be used for the plausibility check. Combinations of several features can also be used for this purpose.

To enable the CAM-AM software to optimally orient and position the digital dental object 100-1 in the dental technical workflow, a classification of the digital dental object 100-1 should be known. In this case, for example, rules for an orientation of the digital dental object can be defined so that an optimized support structure generation can be performed automatically.

If the class, i.e., the indication, of the digital dental object 100-1 is known to the CAD-CAM software, further measures can be taken on this basis to optimize the workflow during production. These measures include, for example, the optimization of an alignment of the digital dental object so that the manufactured dental object requires as few support structures as possible and a large part of the surface of the dental object no longer needs to be reworked.

For this purpose, a digital reference object can be retrieved from a database based on the determined class, for example. For example, if it were recognized that the digital dental object 100-1 is a full denture, a digital reference object of the class “full denture” can be retrieved from the database. The digital dental object 100-1 may then be transformed to the reference object in the coordinate system. For example, an orientation, a position, or a size of the digital dental object 100-1 is adapted to the reference object. In addition, a manufacturing method may be provided for the reference object of the respective class that is used to manufacture the digital dental object.

The recognition method for the dental object 100-1 may be implemented in a manufacturing device 200 that can be used to manufacture the real dental objects 100-2 based on the digital dental objects 100-1, such as a printing device for 3D printing or a milling device. For this purpose, the manufacturing device 200 comprises, for example, a computer having a digital memory and a processor for executing a computer program by which the recognition method is implemented.

All features explained and shown in connection with individual embodiments of the invention may be provided in different combinations in the subject matter of the invention to simultaneously realize their beneficial effects.

All method steps can be implemented by means which are suitable for executing the respective method step. All functions that are executed by objective features can be a method step of a method.

In some embodiments, the innovations may be implemented in diverse general-purpose or special-purpose computing systems. For example, the computing environment can be any of a variety of computing devices (e.g., desktop computer, laptop computer, server computer, tablet computer, gaming system, mobile device, programmable automation controller, etc.) that can be incorporated into a computing system comprising one or more computing devices.

In some embodiments, the computing environment includes one or more processing units and memory. The processing unit(s) execute computer-executable instructions. A processing unit can be a central processing unit (CPU), a processor in an application-specific integrated circuit (ASIC), or any other type of processor. In a multi-processing system, multiple processing units execute computer-executable instructions to increase processing power. A tangible memory may be volatile memory (e.g., registers, cache, RAM), non-volatile memory (e.g., ROM, EEPROM, flash memory, etc.), or some combination of the two, accessible by the processing unit(s). The memory stores software implementing one or more innovations described herein, in the form of computer-executable instructions suitable for execution by the processing unit(s).

A computing system may have additional features. For example, in some embodiments, the computing environment includes storage, one or more input devices, one or more output devices, and one or more communication connections. An interconnection mechanism such as a bus, controller, or network, interconnects the components of the computing environment. Typically, operating system software provides an operating environment for other software executing in the computing environment, and coordinates activities of the components of the computing environment.

The tangible storage may be removable or non-removable, and includes magnetic or optical media such as magnetic disks, magnetic tapes or cassettes, CD-ROMs, DVDs, or any other medium that can be used to store information in a non-transitory way and can be accessed within the computing environment. The storage stores instructions for the software implementing one or more innovations described herein.

The input device(s) may be, for example: a touch input device, such as a keyboard, mouse, pen, or trackball; a voice input device; a scanning device; any of various sensors; another device that provides input to the computing environment; or combinations thereof. The output device may be a display, printer, speaker, CD-writer, or another device that provides output from the computing environment.

The scope of protection of the present invention is given by the claims and is not limited by the features explained in the description or shown in the figures.

### REFERENCE LIST

100-1 Digital dental object
100-2 Dental object to be manufactured
101 Classification data set
103 Class
109 Artificial neural network
111 Layer
113 Neuron
115 Item
117 Cloud of points
200 Manufacturing device

1. A recognition method for a dental object (100-1), comprising the steps:
providing (S101) a digital dental object (100-1) in a coordinate system describing a shape of the dental object (100-2) to be manufactured; and
automatically assigning (S102) the digital dental object (100-1) to a specified class based on the shape by a self-learning algorithm.

2. The recognition method according to claim 1,
wherein a number of points on the surface of the digital dental object (100-1) is detected.

3. The recognition method according to claim 2,
wherein the points on the surface of the digital dental object (100-1) are randomly selected.

4. The recognition method according to claim 2,
wherein coordinates of the detected points form an input for an artificial neural network (109).

5. The recognition method according to claim 4,
wherein the artificial neural network (109) has been trained by a plurality of training data sets.

6. The recognition method according to claim 4,
wherein the class is output by the artificial neural network (109).

7. The recognition method according to claim 1,
wherein a digital reference object is assigned to the digital dental object (100-1) based on the assigned class.

8. The recognition method according to claim 7,
wherein the digital dental object (100-1) is transformed based on the assigned class and/or the reference object in the coordinate system.

9. The recognition method according to claim 1,
wherein a manufacturing method is assigned to the digital dental object (100-1) based on the assigned class.

10. The recognition method according to claim 9,
wherein further spatial structures are added to the digital dental object (100-1) based on the assigned manufacturing method.

11. The recognition method according to claim 9,
wherein the dental object (100-2) is produced by the manufacturing method.

12. The recognition method of claim 11,
wherein the manufacturing method is an additive or subtractive manufacturing method.

13. The recognition method according to claim 1,
wherein a correctness of the assignment is checked by geometric features of the digital dental object (100-1).

14. A manufacturing apparatus (200) for a dental object (100-2), which is adapted to perform the recognition method according to claim 1.

15. A computer program product comprising
program code which is stored on a non-transitory machine-readable medium comprising computer instructions executable by a processor, which computer instructions cause the processor to perform the method according to claim 1.
